(19) United States
(12) Patent Application Publication
(10) Pub. No.: US 2024/0301509 A1
(43) Pub. Date: Sep. 12, 2024

(54) METHODS AND COMPOSITIONS FOR DETERMINING EARLY OR LATE ESTRUS IN GILTS

(71) Applicant: South Dakota Board of Regents, Pierre, SD (US)

(72) Inventors: Jodi Mirissa Morton, Brookings, SD (US); Crystal Lynette Levesque, Brookings, SD (US)

(21) Appl. No.: 18/597,250

(22) Filed: Mar. 6, 2024

Related U.S. Application Data
(60) Provisional application No. 63/488,557, filed on Mar. 6, 2023.

(51) Int. Cl. C12Q 1/6888

(52) U.S. Cl. CPC C12Q 1/6888; C12Q 2600/124; C12Q 2600/158

(57) ABSTRACT

Disclosed are materials and methods for assisting decision making with regard to gilt (young female pigs) management on pig farms. The methods include means for determining which gilts will develop traits of reproductive success, including predicting early or late estrus, and which should be sold prior to becoming overweight for the full value market.

### CROSS-REFERENCE TO RELATED APPLICATIONS

This application claims benefit of U.S. Provisional Application No. 63/488,557, filed Mar. 6, 2023, incorporated herein by reference in its entirety.

### STATEMENT REGARDING FEDERALLY-SPONSORED RESEARCH

This invention was made with government support under Grant No. 2021-67016-33497 awarded by the USDA-NIFA-Agriculture and Food Research Initiative. The government has certain rights in the invention.

### BACKGROUND OF THE INVENTION

The present disclosure relates to methods and materials useful to make decisions in a pig farming business, particularly with regard to replacement gilt selection decisions.

Approximately 20% of gilts (young female pigs) intended as replacement breeding stock never attain puberty (deemed anestrus) or are not successfully bred. The only known predictor of reproductive success is age at puberty, where early puberty is associated with improved long-term reproductive performance and more full value offspring at market.

Puberty can occur over a large range in gilts, between approximately 145 to approximately 228 days old, with an average of approximately 205 days old.

Current on-farm techniques to determine gilts that have achieved puberty rely on human observation to detect changes in gilt behavior at the time of estrus and are not suitable for identification of candidate gilts in the pre-pubertal phase of growth. By the time a gilt has been identified as not suitable for breeding (eg. puberty at >210 days of age, anestrus) she is above the target weight and age for a market hog and must be sold into a cull sow market at a substantially reduced price.

The ability to ‘weed out’ gilts determined to have a low likelihood of reproductive success at an age and weight where the gilt could be finished out as a market hog has the potential to not only save considerable time and effect on the part of barn production staff but also dramatically reduce overall gilt replacement costs.

The present invention provides solutions to the financial and scientific challenges facing the pork industry.

### SUMMARY OF THE INVENTION

Disclosed herein is a panel of markers which can be used to determine the estrus state of the gilt. These markers can be used alone, or can be taken in combination to determine the estrus state. For example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more markers can be used to determine estrus state. These markers include, but are not limited to, GYS2; IFN-alpha 16; LIN28A; ANO2; LOXL2; GREB1; LOC100518848; LOC110261636; LOC100518075; LOC100520618; PRELID3B; LOC100621931; GTSF1; STK32A; LPB; and SPAI2.

Also disclosed herein is a method of determining a gilt is ready to breed, the method comprising: a) identifying a relative change in gene expression in at least one gene associated with growth, reproduction, or immune function in a gilt, b) determining reproductive success likelihood in the gilt; and c) breeding said gilt.

Further disclosed is a method of determining a gilt is anestrus, the method comprising: a) identifying gene expression level in at least one gene associated with growth, reproduction, or immune function in a gilt, b) based on results of step a), determining that anestrus is likely in the gilt; and c) culling or selling the gilt.

Also disclosed herein is a kit for determining reproductive success in a gilt, comprising primers for determining gene expression of at least one gene associated with growth, reproduction, or immune function in a gilt, wherein the genes are selected from one or more genes listed herein. The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects described below. The patent or application file may contain at least one drawing executed in color. Copies of this patent or patent application publication with color drawing(s) will be provided by the Office upon request and payment of the necessary fee.

FIG. 1. Fold change (early vs late) of differentially expressed genes in at least 3 females in either early or late estrus groups at d100, d130, and d160 of age.

FIG. 2. Fold change (late vs early) of differentially expressed genes in at least 3 females in either early or late estrus groups at d100, d130, and d160 of age.

FIG. 3. A. heat map and B. principal component analysis of vaginal transcriptome of gilts that expressed estrus early (160-180 days of age) and late (200-215 days of age). Clustering type: Hierarchical; Distance metric: Euclidean; Linkage method: Centroid (fast); Genes: # data points: 53; # tree nodes: 51; Max tree depth: 12; Experiments: # data points: 2; # tree nodes: 0; Max tree depth: 0.

### DETAILED DESCRIPTION

Terms used throughout this application are to be construed with ordinary and typical meaning to those of ordinary skill in the art. However, Applicant desires that the following terms be given the definition as defined below.

### Terminology

As used in the specification and claims, the singular form “a,” “an,” and “the” include plural references unless the context clearly dictates otherwise. For example, the term “a particle” includes a plurality of particles, including mixtures thereof.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22: 1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The term “increased” or “increase” as used herein generally means an increase by a statistically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

A “label” or a “detectable moiety” is a composition detectable by spectroscopic, photochemical, biochemical, immunochemical, chemical, or other physical means. For example, useful labels include fluorescent dyes, electron-dense reagents, enzymes (for example, as commonly used in an ELISA), biotin, digoxigenin, or haptens and proteins which can be made detectable, e.g., by incorporating a radiolabel into the peptide or used to detect antibodies specifically reactive with the peptide.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C. or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., supra.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications. Academic Press, Inc. N.Y.).

“Antibody” means a protein comprising one or more polypeptides substantially encoded by all or part of the recognized immunoglobulin genes. The recognized immunoglobulin genes, for example in humans, include the kappa (κ), lambda (κ) and heavy chain genetic loci, which together compose the myriad variable region genes, and the constant region genes mu (μ), delta (δ), gamma (γ), epsilon (ε) and alpha (α), which encode the IgM, IgD, IgG, IgE, and IgA isotypes respectively. Antibody herein is meant to include full length antibodies and antibody fragments and may refer to a natural antibody from any organism, an engineered antibody or an antibody generated recombinantly for experimental, therapeutic or other purposes as further defined below. Antibody fragments include Fab. Fab′, F(ab′)2, Fv, scFv or other antigen-binding subsequences of antibodies and can include those produced by the modification of whole antibodies or those synthesized de novo using recombinant DNA technologies. The term “antibody” refers to both monoclonal and polyclonal antibodies. Antibodies can be antagonists, agonists, neutralizing, inhibitory or stimulatory.

Biomarkers can be used in conjunction with other diagnostic tools or used alone.

The term “surrogate marker,” “biomolecular marker,” “biomarker” or “marker” (also sometimes referred to herein as a “target analyte,” “target species” or “target sequence”) refers to a molecule whose measurement provides information as to the state of a subject. Measurements of the biomarker may be used alone or combined with other data obtained regarding a subject in order to determine the state of the subject.

A biomarker may be over-expressed (over-abundant) or under-expressed (under abundant) relative to a control. The biomarker can be an allelic variant, truncated or mutated form of a wild-type nucleic acid or protein. The biomarker can be a splice variant.

A biomarker may be determined to be “differentially present” in a variety of ways, for example, between different phenotypic statuses if the mean or median level (particularly the expression level of the associated mRNAs as described below) of the biomarker in the different groups is calculated to be statistically significant. Common tests for statistical significance include, among others, t-test, ANOVA, Kruskal-Wallis, Wilcoxon, Mann-Whitney and odds ratio.

As described herein, a biomarker may be a nucleic acid or a protein or any and all combinations of these molecules.

In various embodiments, the biomarkers used in the panels of the invention can be detected either as proteins or as nucleic acids (e.g. mRNA or cDNA transcripts) in any combination. In various embodiments, the protein form of a biomarker is measured. As will be appreciated by those in the art, protein assays may be done using standard techniques such as ELISA assays. In various embodiments, the nucleic acid form of a biomarker (e.g., the corresponding mRNA) is measured. In various exemplary embodiments, one or more biomarkers from a particular panel are measured using a protein assay and one or more biomarkers from the same panel are measured using a nucleic acid assay.

In various embodiments, the biomarker is a nucleic acid. The term “nucleic acid” or “oligonucleotide” or grammatical equivalents herein means at least two nucleotides covalently linked together. A nucleic acid of the present invention will generally contain phosphodiester bonds, although in some cases, as outlined below, for example in the use of binding ligand probes, nucleic acid analogs are included that may have alternate backbones.

### Methods of Determining Estrus State of a Gilt

Determining estrus stage in a gilt can have significant economic impacts, as described in the “background” section above. For example, a gilt determined to be in estrus can be bred or sold at a higher value. A gilt which has not yet reached estrus, or which has been determined to be anestrus, can be sold, slaughtered, or culled. Therefore, determining the state of estrus can be used to make decisions and act accordingly. Without the knowledge of the estrus state of the gilt, one would take, or not take, different actions.

Disclosed herein is a panel of markers which can be used to determine the estrus state of the gilt. These markers can be used alone, or can be taken in combination to determine the estrus state. For example, 1, 2, 3, 4, 5, 6, 7, 8, 9, 10, 11, 12, 13, 14, 15, or 16 or more markers can be used to determine estrus state. These markers include, but are not limited to, GYS2; IFN-alpha 16; LIN28A; ANO2; LOXL2; GREB1; LOC100518848; LOC110261636; LOC100518075; LOC100520618; PRELID3B; LOC100621931; GTSF1; STK32A; LPB; and SPAI2.

For example, Table 5 in the “Examples” section shows whether the markers listed above, when higher or lower compared to a reference standard (also referred to herein as “up” or “down”), determine state of estrus. For example, when markers IFN-ALPHA-16, GREB1 and GYS2 are below a reference standard, this can indicate early estrus. When they are above a reference standard, this can indicate late estrus. On the other hand, when markers LIN28A, ANO2, and LOXL2 are above a reference range, this can indicate early estrus. When they are below a reference range, this can indicate late estrus. The reference ranges are provided below, and by “higher” is meant that the amount is above the reference range, by “lower” is meant that the amount is below the reference range, and by “normal” is meant that the number is within a reference range. One can, for example, determine that anything above 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% or more above the reference range is considered “higher” while anything 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% below or more below the reference range is considered “lower.” Table 1 shows various marker ranges.

TABLE 1

| | Reference Range | | |
|---|---|---|---|
| Gene | (Ct) | Above | Below |
| LOC110261636 | 28-32 | Early | Late |
| LOC100518075 | 28-32 | Early | Late |
| LOC100520618 | 28-32 | Early | Late |
| LIN28A | 28-32 | Early | Late |
| LOC100621931 | 28-32 | Early | Late |
| PRELID3B | 28-32 | Early | Late |
| STK32A | 28-32 | Early | Late |
| ANO2 | 28-32 | Early | Late |
| LOXL2 | 28-32 | Early | Late |
| GYS2 | 28-32 | Late | early |
| SPAI-2 | 28-32 | Late | early |
| GTSF1 | 28-32 | Late | early |
| LOC100518848 | 28-32 | Late | early |
| LBP | 28-32 | Late | early |
| GREB1 | 28-32 | Late | early |
| IFN-ALPHA-16 | 28-32 | Late | early |

Based on these findings, disclosed herein is a method of determining whether estrus stage of a gilt at a certain age and acting accordingly, the method comprising: a) obtaining vaginal epithelium cells from a gilt that is at least 80 days old, b) identifying in the cells gene expression of at least one gene associated with growth, reproduction, or immune function in a gilt, and c) determining estrus stage in the gilt, wherein a gilt which has undergone estrus is bred, and a gilt that has not yet undergone estrus is sold or culled.

Again, important here is that, but for the determination of estrus state, one of skill in the art would have taken different action. So if a gilt is determined to be in estrus, and the only way to determine that at an early age (less than 130 days old or 300 lbs, for example) is by using the genetic markers disclosed herein, then that gilt can be bred, whereas without the knowledge of the test, that would not have been the case. Similarly, a gilt determined to have late estrus can be sold, slaughtered, or culled, which would not have been determined at that age/weight without the genetic marker test described herein. Again, similarly, if a gilt is determined to be anestrus, and not likely to ever achieve estrus, that gilt can be culled. But for the knowledge of the assay, that gilt would not have been distinguished from other gilts which are in, or have the potential to be in, estrus.

The assay can be carried out on the gilt at a variety of weights or timepoints. For example, the assay can be carried out when the gilt is from 80-160 days old; 90-140 days old; 100-130 days old; or 110-120 days old, for example. Weight can also be used to determine when the assay is done. For example, the gilt can weight 150-350 lbs, from 200-300 lbs, or from 250-300 lbs, or about 300 lbs. The age and weight can be considered together when deciding when to complete the assay.

In another example, the assay can be carried out at two or more timepoints and/or weights on the same gilt to determine whether the gilt is progressing towards estrus. Progression towards estrus can be determined by determining that a significant change occurs in the markers as compared to a first test. For example, the markers may shift in the direction of estrus as compared to the first test. The markers can change by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% or more compared to the first test. By determining that a change is taking place, one can determine that the gilt is moving towards estrus. On the other hand, if the markers are not changing significantly, for example, by less than 10%, one can determine that the gilt may be anestrus.

Two or more gilts can undergo the testing, and when this occurs, the gilts can be separated based on the results. For example, group of gilts can be sorted with the same early or late estrus determination. These gilts can then be bred, sold, slaughtered, or culled based on the results of the assay.

A gilt can be determined to have a relatively low reproductive potential or a relatively high reproductive potential based on the assay. A gilt can be considered to have relatively low reproductive potential if one or more of the markers are determined to be below the threshold discussed above. On the other hand, a gilt can be considered to have relatively high reproductive value if one or more of the markers are determined to be above the threshold discussed above. Of course, the number of markers which are used can be factored into this equation. For example, if 16 markers are used, one can decide that a gilt has relatively low reproductive potential if 8 or more of the markers indicate such. By way of specific examples, one could determine that if greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the markers indicate that the gilt has relatively low reproductive potential, then the determination could be made that the gilt has low reproductive potential. On the other hand, if greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the markers indicate that the gilt has relatively high reproductive potential, then the determination could be made that the gilt has high reproductive potential. Certain markers can also be weighed more heavily than others based on desired outcome.

Other non-genetic markers can also be considered in making the determination regarding reproductive potential. Examples include, but are not limited to, behavioral; hormonal; and physical traits.

Also disclosed herein is a method of determining a gilt is ready to breed, the method comprising: a) identifying a relative change in gene expression in at least one gene associated with growth, reproduction, or immune function in a gilt, b) determining reproductive success likelihood in the gilt; and c) breeding said gilt.

Further disclosed is a method of determining a gilt is anestrus, the method comprising: a) identifying gene expression level in at least one gene associated with growth, reproduction, or immune function in a gilt, b) based on results of step a), determining that anestrus is likely in the gilt; and c) culling or selling the gilt.

A nucleic acid sample can be obtained from the gilt prior to step a) and used in identifying relative change in gene expression. The sample can be from vaginal mucosa of a gilt. As discussed above, a relative change in gene expression can determined by comparing at least one gene's expression levels during a first testing window at an age less than 100 days old and comparing the at least one gene's expression levels during a second testing window at an age greater than 80 days old but less than 160 days old. The first testing window range can be selected from a group comprising: 30 days old-100 days old; 40 days old-90 days old; 50 days old-80 days old; 60 days old-80 days old; 70 days old-80 days old; and 70 days old-77 days old. The second testing window range can be selected from a group comprising: 80 days old-160 days old; 90 days old-140 days old; 100 days old-130 days old; and 110 days old-120 days old.

Biomarkers generally can be measured and detected through a variety of assays, methods and detection systems known to one of skill in the art. The term “measuring,” “detecting,” or “taking a measurement” refers to a quantitative or qualitative determination of a property of an entity, for example, quantifying the amount or concentration of a molecule or the activity level of a molecule. The term “concentration” or “level” can refer to an absolute or relative quantity. Measuring a molecule may also include determining the absence or presence of the molecule. Various methods include but are not limited to refractive index spectroscopy (RI), ultra-violet spectroscopy (UV), fluorescence analysis, electrochemical analysis, radiochemical analysis, near-infrared spectroscopy (near-IR), infrared (IR) spectroscopy, nuclear magnetic resonance spectroscopy (NMR), light scattering analysis (LS), mass spectrometry, pyrolysis mass spectrometry, nephelometry, dispersive Raman spectroscopy, gas chromatography, liquid chromatography, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, colorimetry and surface plasmon resonance (such as according to systems provided by Biacore Life Sciences). In this regard, biomarkers can be measured using the above-mentioned detection methods, or other methods known to the skilled artisan. Other biomarkers can be similarly detected using reagents that are specifically designed or tailored to detect them.

Different types of biomarkers and their measurements can be combined in the compositions and methods of the present invention. In various embodiments, the protein form of the biomarkers is measured. In various embodiments, the nucleic acid form of the biomarkers is measured. In exemplary embodiments, the nucleic acid form is mRNA. In various embodiments, measurements of protein biomarkers are used in conjunction with measurements of nucleic acid biomarkers.

Methods for detecting mRNA, such as RT-PCR, real time PCR, branch DNA, NASBA and others, are well known in the art. Using sequence information provided by the database entries for the biomarker sequences, expression of the biomarker sequences can be detected (if present) and measured using techniques well known to one of ordinary skill in the art. For example, sequences in sequence database entries or sequences disclosed herein can be used to construct probes for detecting biomarker RNA sequences in, e.g., Northern blot hybridization analyses or methods which specifically, and, preferably, quantitatively amplify specific nucleic acid sequences. As another example, the sequences can be used to construct primers for specifically amplifying the biomarker sequences in, e.g., amplification-based detection methods such as reverse-transcription based polymerase chain reaction (RT-PCR). When alterations in gene expression are associated with gene amplification, deletion, polymorphisms and mutations, sequence comparisons in test and reference populations can be made by comparing relative amounts of the examined DNA. sequences in the test and reference cell populations. In addition to Northern blot and RT-PCR, RNA can also be measured using, for example, other target amplification methods (e.g., TMA, SDA, NASBA), signal amplification methods (e.g., bDNA), nuclease protection assays, in situ hybridization and the like.

In one embodiment in the present invention are biochip assays. By “biochip” or “chip” herein is meant a composition generally comprising a solid support or substrate to which a capture binding ligand (also called an adsorbent, affinity reagent or binding ligand, or when nucleic acid is measured, a capture probe) is attached and can bind either proteins, nucleic acids or both. Generally, where a biochip is used for measurements of protein and nucleic acid biomarkers, the protein biomarkers are measured on a chip separate from that used to measure the nucleic acid biomarkers. In various embodiments, biomarkers are measured on the same platform, such as on one chip. In various embodiments, biomarkers are measured using different platforms and/or different experimental runs.

By “binding ligand,” “capture binding ligand,” “capture binding species,” “capture probe” or grammatical equivalents herein is meant a compound that is used to detect the presence of or to quantify, relatively or absolutely, a target analyte, target species or target sequence (all used interchangeably) and that will bind to the target analyte, target species or target sequence. Generally, the capture binding ligand or capture probe allows the attachment of a target species or target sequence to a solid support for the purposes of detection as further described herein. Attachment of the target species to the capture binding ligand may be direct or indirect. In exemplary embodiments, the target species is a biomarker. As will be appreciated by those in the art, the composition of the binding ligand will depend on the composition of the biomarker. Binding ligands for a wide variety of biomarkers are known or can be readily found using known techniques. For example, when the biomarker is a protein, the binding ligands include proteins (particularly including antibodies or fragments thereof (Fabs, etc.) as discussed further below) or small molecules. The binding ligand may also have cross-reactivity with proteins of other species. Antigen-antibody pairs, receptor-ligands, and carbohydrates and their binding partners are also suitable analyte-binding ligand pairs. In various embodiments, the binding ligand may be nucleic acid. Nucleic acid binding ligands also find particular use when nucleic acids are binding targets.

In various exemplary embodiments, the capture binding ligand is an antibody. These embodiments are particularly useful for the detection of the protein form of a biomarker.

Detecting or measuring the level (e.g. the transcription level) of a biomarker involves binding of the biomarker to a capture binding ligand, generally referred to herein as a “capture probe” when the mRNA of the biomarker is to be detected on a solid support. In that sense, the biomarker is a target sequence. The term “target sequence” or “target nucleic acid” or grammatical equivalents herein means a nucleic acid sequence that may be a portion of a gene, a regulatory sequence, genomic DNA, cDNA, RNA including mRNA and rRNA, or others. As is outlined herein, the target sequence may be a target sequence from a sample, or a secondary target such as a product of an amplification reaction such as PCR, etc. In some embodiments, measuring a nucleic acid can thus refer to measuring the complement of the nucleic acid. It may be any length, with the understanding that longer sequences are more specific.

The target sequence may also comprise different target domains; for example, a first target domain of the sample target sequence may hybridize to a first capture probe, a second target domain may hybridize to a label probe (e.g. a “sandwich assay” format), etc. The target domains may be adjacent or separated as indicated. Unless specified, the terms “first” and “second” are not meant to confer an orientation of the sequences with respect to the 5′-3′ orientation of the target sequence. For example, assuming a 5′-3′ orientation of the target sequence, the first target domain may be located either 5′ to the second domain, or 3′ to the second domain.

When nucleic acids are used as the target analyte, the assays of the invention can take on a number of embodiments. In one embodiment, the assays are done in solution format, using any number of solution based formats. In one embodiment, end-point or real time PCR formats are used, as are well known in the art. These assays can be done either as a panel, in individual tubes or wells, or as multiplex assays, using sets of primers and different labels within a single tube or well. In addition to PCR-based solution formats, other formats can be utilized, including, but not limited to for example ligation based assays utilizing FRET dye pairs. In this embodiment, only upon ligation of two (or more) probes hybridized to the target sequence is a signal generated.

In many embodiments, the assays are done on a solid support, utilizing a capture probe associated with the surface. As discussed herein, the capture probes (or capture binding ligands, as they are sometimes referred to) can be covalently attached to the surface, for example using capture probes terminally modified with functional groups, for example amino groups, that are attached to modified surfaces such as silanized glass. Alternatively, non-covalent attachment, such as electrostatic, hydrophobic/hydrophilic adhesion can be utilized. As is appreciated by those in the art and discussed herein, a large number of attachments are possible on a wide variety of surfaces,

In this embodiment, the assays can take on a number of formats. In one embodiment, the target sequence comprises a detectable label, as described herein. In this embodiment, the label is generally added to the target sequence during amplification of the target in one of two ways: either labeled primers are utilized during the amplification step or labeled dNTPs are used, both of which are well known in the art. The label can either be a primary or secondary label as discussed herein. For example, in one embodiment, the label on the primer and/or a dNTP is a primary label such as a fluorophore. Alternatively, the label may be a secondary label such as biotin or an enzyme; for example, in one embodiment, the primers or dNTPs are labeled with biotin, and then a streptavidin/label complex is added. In one embodiment, the streptavidin/label complex contains a label such as a fluorophore. In an alternative embodiment, the streptavidin/label complex comprises an enzymatic label. For example, the complex can comprise horseradish peroxidase, and upon addition of TMB, the action of the horseradish peroxidase causes the TMB to precipitate, causing an optically detectable event. This has a particular benefit in that the optics for detection does not require the use of a fluorimeter.

In alternate embodiments, the solid phase assay relies on the use of a labeled soluble capture ligand, sometimes referred to as a “label probe” or “signaling probe” when the target analyte is a nucleic acid. In this format, the assay is a “sandwich” type assay, where the capture probe binds to a first domain of the target sequence and the label probe binds to a second domain. In this embodiment, the label probe can also be either a primary (e.g. a fluorophore) or a secondary (biotin or enzyme) label. In one embodiment, the label probe comprises biotin, and a streptavidin/enzyme complex is used, as discussed herein. As above, for example, the complex can comprise horseradish peroxidase, and upon addition of TMB, the action of the horseradish peroxidase causes the TMB to precipitate, causing an optically detectable event.

Detection of a target species in some embodiments requires a “label” or “detectable marker” (as described below) that can be incorporated in a variety of ways. Thus, in various embodiments, the composition comprises a “label” or a “detectable marker.” In one embodiment, the target species (or target analyte or target sequence) is labeled; binding of the target species thus provides the label at the surface of the solid support.

In embodiments finding particular use herein, a sandwich format is utilized, in which target species are unlabeled. In these embodiments, a “capture” or “anchor” binding ligand is attached to the detection surface as described herein, and a soluble binding ligand (frequently referred to herein as a “signaling probe,” “label probe” or “soluble capture ligand”) binds independently to the target species and either directly or indirectly comprises at least one label or detectable marker.

By “label” or “labeled” herein is meant that a compound has at least one molecule, element, isotope or chemical compound attached to enable the detection of the compound. In general, labels fall into four classes: a) isotopic labels, which may be radioactive or heavy isotopes: b) magnetic, electrical, thermal; c) colored or luminescent dyes; and d) enzymes; although labels include particles such as magnetic particles as well. The dyes may be chromophores or phosphors but are preferably fluorescent dyes, which due to their strong signals provide a good signal-to-noise ratio for decoding. Suitable dyes for use in the invention include, but are not limited to, fluorescent lanthanide complexes, including those of Europium and Terbium, fluorescein, rhodamine, tetramethylrhodamine, cosin, crythrosin, coumarin, methyl-coumarins, pyrene, Malacite green, stilbene, Lucifer Yellow, Cascade Blue, Texas Red, Alexa dyes and others described in the 6th Edition of the Molecular Probes Handbook by Richard P. Haugland, hereby expressly incorporated by reference. Additional labels include nanocrystals or Q-dots as described in U.S. Pat. No. 6,544,732 incorporated by reference.

In various embodiments, a secondary detectable label is used. A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, etc. Secondary labels include, but are not limited to, one of a binding partner pair; chemically modifiable moieties; nuclease inhibitors, enzymes such as horseradish peroxidase, alkaline phosphatases, lucifierases, etc. Secondary labels can also include additional labels.

In various embodiments, the secondary label is a binding partner pair. For example, the label may be a hapten or antigen, which will bind its binding partner. For example, suitable binding partner pairs include, but are not limited to: antigens (such as proteins (including peptides)) and antibodies (including fragments thereof (Fabs, etc.)); proteins and small molecules, including biotin/streptavidin; enzymes and substrates or inhibitors; other protein-protein interacting pairs; receptor-ligands; and carbohydrates and their binding partners. Nucleic acid-nucleic acid binding proteins pairs are also useful. In general, the smaller of the pair is attached to the NTP for incorporation into the primer. Preferred binding partner pairs include, but are not limited to, biotin (or imino-biotin) and streptavidin, digeoxinin and Abs, and Prolinx™ reagents.

In the sandwich formats of the invention, an enzyme serves as the secondary label, bound to the soluble capture ligand. Of particular use in some embodiments is the use of horseradish peroxidase, which when combined with 3,3′,5,5′-tetramethylbenzidine (TMB) forms a colored precipitate which is then detected. In some cases, the soluble capture ligand comprises biotin, which is then bound to a enzyme-streptavidin complex and forms a colored precipitate with the addition of TMB.

In various embodiments, the label or detectable marker is a conjugated enzyme (for example, horseradish peroxidase). In various embodiments, the system relies on detecting the precipitation of a reaction product or on a change in, for example, electronic properties for detection. In various embodiments, none of the compounds comprises a label.

As used herein, the term “fluorescent signal generating moiety” or “fluorophore” refers to a molecule or part of a molecule that absorbs energy at one wavelength and re-emits energy at another wavelength. Fluorescent properties that can be measured include fluorescence intensity, fluorescence lifetime, emission spectrum characteristics, energy transfer, and the like.

Signals from single molecules can be generated and detected by a number of detection systems, including, but not limited to, scanning electron microscopy, near field scanning optical microscopy (NSOM), total internal reflection fluorescence microscopy (TIRFM), and the like.

Thus, a detection system for fluorophores includes any device that can be used to measure fluorescent properties as discussed above. In various embodiments, the detection system comprises an excitation source, a fluorophore, a wavelength filter to isolate emission photons from excitation photons and a detector that registers emission photons and produces a recordable output, in some embodiments as an electrical signal or a photographic image. Examples of detection devices include without limitation spectrofluorometers and microplate readers, fluorescence microscopes, fluorescence scanners (including e.g. microarray readers) and flow cytometers.

In various exemplary embodiments, the binding of the biomarker to the binding ligand is specific or selective, and the binding ligand is part of a binding pair. By “specifically bind” or “selectively bind” or “selective for” a biomarker herein is meant that the ligand binds the biomarker with specificity sufficient to differentiate between the biomarker and other components or contaminants of the test sample.

The term “solid support” or “substrate” refers to any material that can be modified to contain discrete individual sites appropriate for the attachment or association of a capture binding ligand. Suitable substrates include metal surfaces such as gold, electrodes, glass and modified or functionalized glass, plastics (including acrylics, polystyrene and copolymers of styrene and other materials, polypropylene, polyethylene, polybutylene, polycarbonate, polyurethanes. Teflon, derivatives thereof, etc.), polysaccharides, nylon or nitrocellulose, resins, mica, silica or silica-based materials including silicon and modified silicon, carbon, metals, inorganic glasses, fiberglass, ceramics, GETEK (a blend of polypropylene oxide and fiberglass) and a variety of other polymers. Of particular use in the present invention are the ClonDiag materials described below.

Frequently, the surface of a biochip comprises a plurality of addressable locations, each of which comprises a capture binding ligand. An “array location,” “addressable location,” “pad” or “site” herein means a location on the substrate that comprises a covalently attached capture binding ligand. An “array” herein means a plurality of capture binding ligands in a regular, ordered format, such as a matrix. The size of the array will depend on the composition and end use of the array. Arrays containing from about two or more different capture binding ligands to many thousands can be made. Generally, the array will comprise 3, 4, 5, 6, 7 or more types of capture binding ligands depending on the end use of the array. In the present invention, the array can include controls, replicates of the markers and the like. Exemplary ranges are from about 3 to about 50. In some embodiments, the compositions of the invention may not be in array format; that is, for some embodiments, compositions comprising a single capture ligand may be made as well. In addition, in some arrays, multiple substrates may be used, either of different or identical compositions. Thus for example, large arrays may comprise a plurality of smaller substrates.

Accordingly, in one aspect, the invention provides a composition comprising a solid support comprising a capture binding ligand for each biomarker of a biomarker panel. In various embodiments, the capture ligand is a nucleic acid. In various embodiments, the capture binding ligand is an antibody. In various embodiments, the composition further comprises a soluble binding ligand for each biomarker of a biomarker panel.

A number of different biochip array platforms as known in the art may be used. For example, the compositions and methods of the present invention can be implemented with array platforms such as GeneChip® (Affymetrix), CodeLink™ Bioarray (Amersham), Expression Array System (Applied Biosystems), SurePrint microarrays (Agilent), Sentrix® LD BeadChip or Sentrix® Array Matrix (Illumina) and Verigene (Nanosphere).

In various exemplary embodiments, detection and measurement of biomarkers utilizes colorimetric methods and systems in order to provide an indication of binding of a target analyte or target species. In colorimetric methods, the presence of a bound target species such as a biomarker will result in a change in the absorbance or transmission of light by a sample or substrate at one or more wavelengths. Detection of the absorbance or transmission of light at such wavelengths thus provides an indication of the presence of the target species.

A detection system for colorimetric methods includes any device that can be used to measure colorimetric properties as discussed above. Generally, the device is a spectrophotometer, a colorimeter or any device that measures absorbance or transmission of light at one or more wavelengths. In various embodiments, the detection system comprises a light source; a wavelength filler or monochromator; a sample container such as a cuvette or a reaction vial; a detector, such as a photoresistor, that registers transmitted light; and a display or imaging element.

Those skilled in the art will be familiar with numerous additional immunoassay formats and variations thereof which may be useful for carrying out the method disclosed herein. See generally E, Maggio, Enzyme-Immunoassay, (CRC Press, Inc., Boca Raton, Fla., 1980); see also U.S. Pat. Nos. 4,727,022; 4,659,678; 4,376,110; 4,275,149; 4,233,402; and 4,230,767.

In general, immunoassays carried out in accordance with the present invention may be homogeneous assays or heterogeneous assays. In a homogeneous assay the immunological reaction usually involves the specific antibody (e.g., anti-biomarker protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays include immunoblotting, immunofluorescence methods, immunoprecipitation, chemiluminescence methods, electrochemiluminescence (ECL) or enzyme-linked immunoassays.

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., 35S, 125I, 131I), enzyme labels (e.g., horseradish peroxidase, alkaline phosphatase), and fluorescent labels (e.g., fluorescein, Alexa, green fluorescent protein, rhodamine) in accordance with known techniques.

Using any of the methods and compositions described herein, a sample can be assayed to determine levels of a biomarker panel. Thus, in one aspect, the invention provides a method of assaying a sample from a patient to determine concentrations of a biomarker panel in the sample. In some embodiments, the method comprises contacting the sample with a composition comprising a solid support comprising a capture binding ligand or capture probe for each biomarker of a biomarker panel.

Any combination of the biomarkers described herein is used to assemble a biomarker panel, which is detected or measured as described herein. As is generally understood in the art, a combination may refer to an entire set or any subset or subcombination thereof. The term “biomarker panel,” “biomarker profile,” or “biomarker fingerprint” refers to a set of biomarkers. As used herein, these terms can also refer to any form of the biomarker that is measured. Specifically, the detection of a plurality of biomarkers in a sample can increase the sensitivity and/or specificity of the test. Thus, in various embodiments, a biomarker panel may include 1, 2, 3, 4, 5, 6, 7, 8, 9, 10, 11, 12, 13, 14, 15, 16, 17, or more types of biomarkers. In various exemplary embodiments, the biomarker panel consists of a minimum number of biomarkers to generate a maximum amount of information. Thus, in various embodiments, the biomarker panel consists of 1, 2, 3, 4, 5, 6, 7, 9, 10, 11, 12, 13, 14, 15, 16, 17, or more types of biomarkers. Where a biomarker panel “consists of” a set of biomarkers, no biomarkers other than those of the set are present. In exemplary embodiments, the biomarker panel consists of 1 biomarker disclosed herein. In various embodiments, the biomarker panel consists of 2 biomarkers disclosed herein. In various embodiments, the biomarker panel consists of 3 biomarkers disclosed herein. In various embodiments, the biomarker panel consists of 4 or more biomarkers disclosed herein.

The invention further provides kits. Specifically disclosed herein is a kit for determining reproductive success in a gilt, comprising primers for determining gene expression of at least one gene associated with growth, reproduction, or immune function in a gilt, wherein the genes are selected from one or more genes listed herein. The kit can further comprise components for nucleic acid amplification and/or detection. This can include a visual indicator. It can be a rapid test for on-site use. The kit can comprise a number of sets of primers selected from the group consisting of: one or more sets of primers; two or more sets of primers; three or more sets of primers; four or more sets of primers; five or more sets of primers; and six or more sets of primers, seven or more sets of primers, eight or more sets of primers, 9 or more sets of primers, 10 or more sets of primers, 11 or more sets of primers, and 12 or more sets of primers. The kit can comprise a reference resource specifying standard values for correlating gene expression with reproductive success in a gilt. Also disclosed is a means for collecting a sample from a gilt.

Kits may comprise a carrier, such as a box, carton, tube or the like, having in close confinement therein one or more containers, such as vials, tubes, ampoules, bottles, pouches, envelopes and the like. In various embodiments, the kits comprise one or more components selected from one or more media or media ingredients and reagents for the measurement of the various biomarkers and biomarker panels disclosed herein. For example, kits of the invention may also comprise, in the same or different containers, one or more DNA polymerases, one or more primers, one or more suitable buffers, one or more nucleotides (such as deoxynucleoside triphosphates (dNPTs) and preferably fluorescently labeled dNTPs) and labeling components. The one or more components may be contained within the same container or may be in separate containers to be admixed prior to use. The kits of the present invention may also comprise one or more instructions or protocols for carrying out the methods of the present invention. In various embodiments, the kit comprises a biomarker assay involving a lateral-flow-based rapid test with detection of risk thresholds, or a biochip with quantitative assays for the constituent biomarkers

Unless defined otherwise, all technical and scientific terms used herein have the same meanings as commonly understood by one of skill in the art to which the disclosed invention belongs.

Those skilled in the art will recognize or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1. Materials & Methods

Animal procedures were reviewed and approved by the South Dakota State University Institutional Animal Care and Use Committee (approval number 19-006A). The original planned sample collection timeline is shown in Table 2. The intent was to complete the listed collections/observations using 30 pre-pubertal gilts in a single cohort. However, due to unforeseen circumstances that occurred with the initial group of 30 gilts (deemed cohort 1), another group of 12 gilts were included (deemed cohort 2), and an additional 29 females (deemed cohort 3) were utilized.

### Cohort 1

D70 collection. A total of 30 pre-pubertal replacement females (PIC 1050; 22.52±1.39 kg; d70 of age) from a single source were housed at South Dakota State University Swine Education and Research Facility (SDSU SERF) in 2 pens (n=15/pen) with greater than 0.92 m²/pig. Gilts were provided a common diet in 5 phases (budget/pig/phase: with ad libitum feed intake and nutrient specifications for prepubertal females as recommended by NRC (2012), as well as ad libitum water access with 1 nipple/cup waterer per pen.

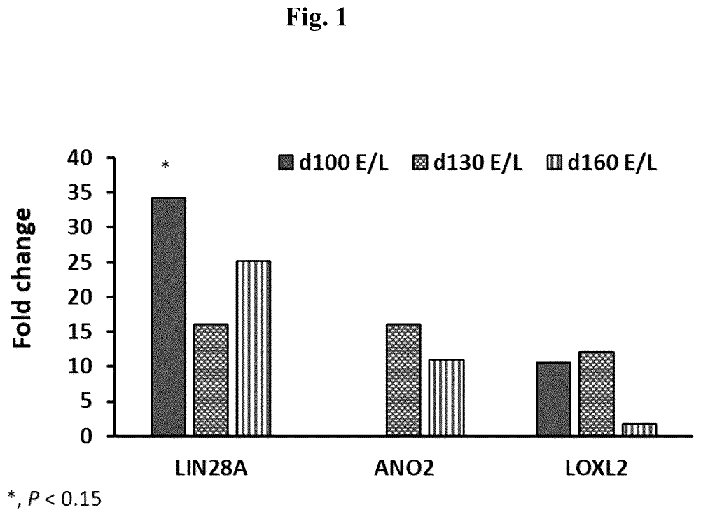

Upon arrival, bodyweight (BW), a 3 mL blood sample [jugular venipuncture (20 g×1.5 inches) collected in serum vacutainer (BD Vacutainer, Franklin Lakes, NJ) and a vaginal swab was collected using a Cervex-Brush cervical cell sampler (Fisherbrand #1437256, Fisher Scientific, Waltham, MA) placed approximately 4 cm into the vagina and then using slight pressure and 3 partial hand rotations, tissue was collected from the dorsal portion and sides to avoid the suburethral diverticulum (FIG. 1). The cytology brush was then placed in a cryovial containing TRIzol reagent (Invitrogen, Waltham, MA), swished for 30 seconds, and then flash frozen in liquid nitrogen on-farm. Following transport to campus, samples were placed in a −80° C. freezer prior to RNA extraction. Blood samples were left at room temperature and allowed to clot before centrifugation (2,500 rpm for 15 minutes at 22° C.), after which serum was removed and stored in microcentrifuge tubes at −20° C. until further analysis.

D100 collection. The original intent of the collection planned for d100 was to collect serial blood samples via ear vein catheters from all 30 gilts and establish a subset of gilts with ‘high’ and ‘low’ levels of luteinizing hormone (LH) based on serum concentrations of LH. As outlined in the timeline, the goal was to begin collecting blood samples at 0700 h, with collections every 30 minutes for 10 hours (0700-1600). The subsets of ‘high LH’ and ‘low LH’ gilts were expected to represent females likely to express an ‘early/average’ estrus or a ‘late’ estrus cycle, respectively. Attempts to place ear-vein catheters into gilts were unsuccessful. Temporary jugular catheters were explored as an alternate method for serial blood sampling; however, due to a lack of availability from manufacturers, supplies required for the protocol were delayed and only 9 gilts of the original group of 30 were selected for serial blood draws. Body weight was used as the selection criteria to attempt to choose gilts likely to express estrus at different ages where 5 of the heaviest gilts and 4 of the lightest gilts were selected from the group of 30 and assumed to represent ‘early’ and ‘late’ estrus gilts, respectively. Two gilts died within 4 h of catheterization. For the remaining 7 catheterized gilts, serial blood samples were collected every 30 minutes for 10 hours to determine serum concentration of LH. Despite challenges with blood collection, d100 vaginal tissue samples were collected as described previously on all 28 remaining in this cohort.

D130 collection. On d130 of age all gilts were again weighed, a vaginal swab collected, and AGD measurements recorded. During vaginal swab collection on one gilt, the swab tip separated from the handle and remained in the vagina. Repeated attempts were made to retrieve the tip without success, so the gilt was removed from test and euthanized using a captive bolt gun. This gilt was one of the 6 from which serial blood samples were collected at d100. Following the d130 collection, only the 5 gilts remaining from the d100 serial blood collection were retained for the remainder of the experiment.

D133, 136, and 139 collections. A single blood draw was collected every 3 days from d133 to 139 from the 5 selected gilts and analyzed for progesterone using radioimmunoassay (RIA; Jolitz et al., 2015). Progesterone (P0130; Sigma Life Science, St. Louis, MO) was used to confirm that the gilts were still pre-pubertal and had not achieved an early estrus.

D143 and 173 collections. At d143 of age an indwelling catheter with a subcutaneous titanium port (Levesque et al., 2011) was implanted in the 3 heaviest gilts (gilt ID 0691, 0684, and 0698) and deemed ‘ported’ gilts. Gilts were allowed to recover from surgery for at least 7 days. Surgery for the two remaining gilts was delayed because they were deemed excessively light BW (0697: 54 kg; 0699: 52 kg). One of these two gilts (gilt ID 0699) was successfully catheterized on D173. By d173, the fifth gilt was still deemed too light (78 kg) and was removed from the experiment and culled.

Boar Exposure. On d160 of age, a single blood draw and a vaginal swab occurred on the 3 “heavy” ported gilts. Of the total 4 ports that were placed, patency was lost for one gilt within a week of the surgery (gilt ID 0691) and one gilt (gilt ID 0684) appeared to suffer from severe anxiety to touch following the surgery such that accessing the port was not feasible. Protocols for post-surgical daily animal care, approved by the University IACUC, were followed and no indications of adverse reaction to surgical procedures (i.e., drugs used, recovery from anesthesia) was noted for this gilt so a justification for the change in behavior is unknown. Per the original collection timeline proposed in Table 2, daily serial blood collection was attempted in gilt ID 0698 from d160-174. Indwelling port was accessed using sterile procedures (Appendix 1) at 0700, with a blood sample collected every 20 minutes for 4 hours. Huber needles were removed at the completion of the daily collection and reinserted the following morning. However, after completion of the first two weeks of bleeding, it became apparent that excessive scar tissue was developing in the skin surrounding the ports. The decision was made to change serial blood collection to every three days, with a 7-day rest period between every two-week bleeding interval. Therefore, serial blood collection began again with gilt IDs 0698 and 0699 on d181, 184, 187, 190, 193 and on d203, 206, 209, 212, and 215, following a 7-day rest period. On blood collection days during boar exposure, serial samples were collected beginning one hour before boar exposure then every 20 minutes up to 3 hours following the completion of boar exposure. Boar exposure occurred every day from 0800-0815 h. Two mature boars, housed at the SERF separate from the gilts, were alternated daily. Additionally, photos of each vulva were taken daily as another way to document estrus based on color change or mucus discharge. Photos were taken daily until standing estrus or d215, whichever came first. Standing estrus was detected by back pressure and human identification of estrus behavior (i.e., ‘locked’ stance, ears erect, grunting, swollen, pink vulva). Once standing estrus was detected a vaginal swab and AGD measurements were recorded. When standing estrus was detected, ports were accessed, and serial blood samples were collected every 2 hours for a total of 96 hours following estrus detection to characterize individual gilt LH levels and subsequent ovulation surge. A single blood sample via jugular venipuncture was then collected at d 8 post-standing estrus to confirm that ovulation did occur based on serum progesterone concentrations. Of the selected 5 gilts where standing estrus was not detected by d215, vaginal swabs were collected. Further, at d223, a single blood sample was collected via jugular venipuncture to confirm the lack of serum concentration of progesterone as a marker of anestrus. Detectable serum progesterone is indicative of an undetected or ‘silent’ heat. Gilts that exhibited estrus, or where circulating levels of progesterone in serum was detected, were retained in the herd based on BW and herd replacement needs. The barn protocol to synchronize gilts for entrance into the breeding herd using Estrumate (Merck Animal Health, Worthington, MN) was applied. Gilts that did not exhibit standing estrus or did not have detectable serum concentration of progesterone after the completion of the trial were deemed ‘anestrus’ and shipped to a cull market.

### Cohort 2

D77 collection. Twelve pre-pubertal replacement females (PIC 1050; 39.17 kg±2.72; d77 of age) were offered the same diet regimen as described for cohort 1. However, due to availability of gilts when the order was placed from the multiplier, these gilts arrived at 11 weeks of age. Therefore, the collections outlined for cohort 2 occurred on d77, 110, 130, 160 and during boar exposure. Upon arrival at d77, gilts were weighed vaginal swabs collected.

D100 collection. At d100 of age, temporary jugular catheters were again attempted. However, within the first two gilts where catheter insertion was attempted, one died following the catheterization procedure. Necropsy results were inconclusive as to cause of death, and thus due to a lack of clear explanation for gilt deaths during the temporary jugular catheterizations, it was decided that serial blood collection for characterization of LH would not be continued. As with the previous cohort, LH characterization could not be used to establish ‘high’ and ‘low’ subsets, thus BW was again used.

D110 collection. On d 110, a single blood draw from all remaining gilts (n=11) was obtained for circulating hormone levels of estradiol and a vaginal swab was obtained.

The collections that occurred on d130, 133, 136, 139, 160 and throughout boar exposure are the same as outlined for cohort 1. For cohort 2, it was decided that the heaviest 9 gilts remaining at d130 would be retained for the remainder of the experiment and would receive indwelling titanium ports. Selected gilts received an indwelling catheter and port on d141±2. A single blood sample was collected from each gilt on D153 and D157 to check for progesterone prior to boar exposure. Based on detectable serum progesterone concentration, it was determined that 2 gilts had achieved estrus prior to start of boar exposure at D160.

### Cohort 3

D74 collection. Thirty pre-pubertal replacement females (PIC 1050; 31.4 kg±4.9; d70 of age) and were offered the same diet regimen as described for cohort 1. One female died within 48 hours of on-farm arrival, and the necropsy determined transport stress was likely cause of death. The collections that occurred on d74 and 100 were the same as described for cohort 1 and 2.

D110 and 120 collections. Gilt BW and vaginal swabs were collected from 29 females.

D130 collection. The collections that occurred on d130 were the same as described above.

D144, 147, 150, 153, 157 collections. Unlike the first two cohorts, no females in cohort 3 received an indwelling titanium port and cephalic vein catheter. A single blood draw was collected every 3 days from d144 to 157 and analyzed for progesterone using RIA to confirm gilts were still pre-pubertal and had not achieved an early estrus.

D159 collection. This collection followed the same protocol as described for d160 collection in previous cohorts. Daily boar exposure began and continued until d213. However, due to an outbreak of PRRSv that occurred near the end of boar exposure the entire herd, including these gilts was depopulated thus no specific evidence of successful breeding was possible in these females.

### Example 2. Laboratory Analysis

### Laboratory Analyses

Estradiol-17β. Serum concentrations of estradiol-17β (E2) were determined in duplicate in all blood samples by RIA (Jolitz et al., 2015). Estradiol-17β (Sigma Life Sciences, St. Loius, MO) was the standard and radioiodinated E2 (MP Biomedicals, Solon, OH) was the tracer. Anitsera (Fort Collins, CO) was used at a dilution of 1:425,000. Sera (300 μl) was extracted with a 4 mL volume of methyl tert-butyl ether. Inhibition curves of increasing amounts of sample were parallel to standard curve.

Luteinizing Hormone. Serum concentrations of luteinizing hormone (LH) were determined in duplicate by RIA. Porcine LH (National Hormone and Peptide Program, NIDDK) was used as the radioidodinated antigen and standard. Luteinizing hormone antiserum (National Hormone and Peptide Program, NIDDK) was used at a dilution of 1:200,000. Inhibition curves of increasing amounts of sample were parallel to standard curve.

Progesterone. Serum concentrations of progesterone were determined in duplicate for all blood samples by RIA (Jolitz et al., 2015). Progesterone (Sigma Life Science, St. Loius MO) was the standard and radioiodinated progesterone (MP Biomedical, Solon, OH) was used as the tracer. Antisera was used at a dilution of 1:700,000. Inhibition curves of increasing amounts of sample were parallel to standard curves.

Extraction of RNA. The cryovials containing vaginal sample and TRIzol reagent were thawed on ice. When nearly thawed, samples were titrated with a needle and syringe 6 times. The sample was then placed into a 15 mL conical tube and 600 μl of chloroform were added to each tube. Samples were shaken and centrifuged (4,500×g for 30 minutes at 4° C.), supernatant was transferred to a 15 mL conical tube, where another 200 μl of chloroform was added. After the second centrifugation, the supernatant was transferred to final 15 mL conical tube. RNA was purified according to the followed protocol. Equal parts 70% ethanol and extracted sample were added to the 15 mL conical tube and vortexed. Then, 700 μl of sample was pipetted into a spin cartridge with collection tube. The sample was centrifuged at 12,000×g for 1 minute at room temperature (25° C.). The flow through was discarded and the process repeated until the entire sample was processed; 700 μl of Wash Buffer 1 was added to the spin cartridge and the sample was again centrifuged at 12,000×g for 1 minute at room temperature. The spin cartridge was placed into a new collection tube and 500 μl of Wash Buffer II with ethanol was added to each collection tube. Samples were then centrifuged at 12,000×g for 1 minute at room temperature. The flow through was discarded and the process was repeated. To dry the membrane, the sample was centrifuged at 12,000×g for 2 minutes. The collection tube was discarded, and the spin cartridge was placed into a recovery tube. A total of 30 μl of DNase/RNase free water was added to each sample tube and the samples were incubated for 1 minute. The samples were centrifuged at 12,000×g for 2 minutes at room temperature, the spin cartridge was removed, and the recovery tube labelled and stored at −80° C. until further analysis. Concentration of RNA was determined via spectrophotometer (Nanodrop, Thermo Scientific, Washington, DE). Purity of RNA was determined by measuring the A260/A280 ratio; purity of RNA for all samples fell within a range of 1.8 to 2.0. Two micrograms of the resulting RNA was reverse transcribed into cDNA using the HighCapacity cDNA Reverse Transcription Kit (Applied Biosystems, Foster City, CA) following the manufacturer's protocol. Previously published mRNA sequences for the genes of interest and porcine RPLP0 (housekeeping gene) were used to design specific forward and reverse primers. Primers were designed using software provided by Integrated DNA Technologies (IDT, Coralville, IA).

Real-Time PCR. Real-time semi-quantitative PCR was used to measure quantity of estrogen receptor-α (ER-α), insulin-like growth factor-1 (IGF-1), tachykinin-3 (TAC-3), and toll-like receptor 4 and 5 (TLR-4 and TLR-5, respectively) vaginal mRNA relative to the amount of porcine RPLP0 mRNA in each sample. Measurements of the relative quantity of the cDNA of interest was carried out using RT2 Real-Time™ SYBR Green/ROX PCR Master Mix (SuperArray Bioscience Corp., Foster City, CA). Twenty microliter reactions were measured using the Stratagene MX3005P quantitative real-time PCR instrument (Agilent Technologies, Foster City, CA). Thermal cycling conditions recommended by the manufacturer (40 cycles of 30 sec at 95° C., 1 min at 55° C., and 1 min at 72° C.) were used for all genes. Concentrations of forward and reverse primers used for the genes of interest and RPLP0 were 300 nM. A linear response was obtained when these concentrations of primer pairs were used with increasing amounts of cDNA. Dissociation curve analysis was performed after each real-time PCR run to confirm that a single amplicon was present. Additionally, all amplicons were electrophoresed through a 2% agarose gel and stained with ethidium bromide to visualize that only amplicons of the appropriate size were present in each sample.

RNA sequencing. A total of 30 RNA samples were sent to a commercial sequencing facility (MRDNA; Shallowater, TX). A total of 5 ‘early’ females and 5 ‘late or anestrus’ females at 100, 130, and 160 d of age were sent for analysis. The sequencing facility performed bulk sequencing, consisting of >600 million reads, and 2×150 base pairs.

Statistical Analysis. All analyses were performed in RStudio (v 4.0.2). Anogenital length measurements were analyzed using the Correlations package in RStudio. Relative gene expression of the analyzed vaginal epithelium was performed using the PCR package. Hormone data was analyzed using descriptive statistics in excel. Analysis of RNA sequencing data utilized the Welch two-sample t test.

### Example 3. Results

Cohort 1 and 2. Ten gilts were determined to have successfully achieved estrus (Table 3) based on physical signs of standing estrus and/or detectable serum concentration of progesterone.

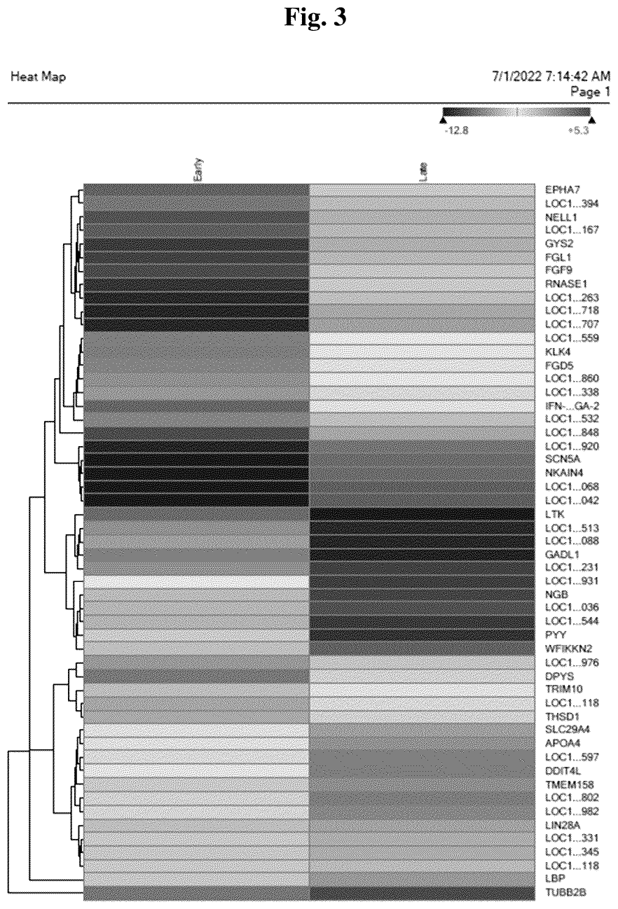

Gene expression. Relative expression of 4 genes associated with growth and reproduction from vaginal tissue collected from 13 animals across 5 timepoints (FIG. 3). Values represent relative fold change from gene expression at d 70/77 of age. At d 160, there was a 9.02-fold increase in relative IGF-1 expression (P=0.02), a 2.57-fold tendency increase (P=0.08) in ER-α expression, and a 7.88-fold increase (P=0.04) in TAC-3 expression. Expression of TLR-4 and TLR-5 were not detected, or lowly detected, in all samples at d 70, d 100, d 130 and d 160 (n=0/13, 3/13, 2/13, and 2/13 samples). At standing estrus or end of trial, expression of TLR-4 and 5 was readily detected in 8/13 samples.

Cohort 3. Across all time points, 27,302 genes were expressed between ‘early’ and ‘late’ estrus gilts. To narrow the pool of target predictor genes, a 4-step selection process was used 1) all genes that were differentially expressed at least 2-fold greater in early or in late estrus gilts across all time points (2,355 genes), 2) all genes that were differentially expressed at least 20-fold greater at any one time point in early or late estrus gilts, 3) all genes at least 10-fold greater in at least 2 time points in early or late estrus gilts and 4) known genes at least 10-fold greater in at least 2 time points in early or late estrus gilts. This process resulted in a list of 11 candidate biomarkers of which 5 were unidentified genes (Table 4). To further narrow the potential target biomarkers a 3-step selection process was used 1) expressed in a minimum of 3 females in 1 group at 1 timepoint, 2) known genes with a minimum of 3 females in 1 groups at d100 or d130, 3) known genes at both d100 and d130. Multiplex PCR on another set of early and late estrus females is underway to further confirm differences in expression of the 6 candidate biomarker genes at d100 and 130 (Table 5).

TABLE 2
Collection timeline.

| Reproductive | Day of | Sample | Blood | |
|---|---|---|---|---|
| development | age | type/activity | analysis | Collection span |
| Prepubertal | 70 | Blood, AGD, | Estradiol, | Single blood draw, AGD measure, and |
| | | vaginal tissue | testosterone, | vaginal swab |
| | | | LH | |
| | 100 | Blood, AGD, | LH | Serial using non-permanent jugular |
| | | vaginal tissue | | catheter, every 30 minutes for 10 h, |
| | | | | single AGD measure and vaginal swab |
| | 130-139 | Blood, AGD, | Progesterone | Single draw every 3 d, single AGD |
| | | vaginal tissue | | measure and vaginal swab at d130 |
| | 140-145 | Implantation of | | |
| | | indwelling | | |
| | | jugular catheter | | |
| Puberty/ | 160-174 | Start of boar | | Daily for 15 minutes |
| standing | | exposure | | |
| estrus | | Blood, AGD, | LH | Blood daily: serial sampling using |
| | | vaginal swab | | indwelling catheter, every 20 min |
| | | | | beginning 60 min prior to boar |
| | | | | exposure until 3 h after boar exposure; |
| | | | | single AGD and vaginal swab at d160 |
| | | | | and again at day standing estrus is |
| | | | | observed |
| | | Blood | Progesterone | Gilts observed in standing estrus only, |
| | | | | single draw collected 8 d after standing |
| | | | | estrus detected |
| | 181-195 | Start of boar | | Daily for 15 minutes |
| | | exposure | | |
| | | Blood, AGD | LH | Blood daily, serial sampling using |
| | | | | indwelling catheter, every 20 min |
| | | | | beginning 60 min prior to boar |
| | | | | exposure until 3 h after boar exposure; |
| | | | | single AGD and vaginal swab at d160 |
| | | | | and again at day standing estrus is |
| | | | | observed |
| | | Blood | Progesterone | Gilts observed in standing estrus only, |
| | | | | single draw collected 8 d after standing |
| | | | | estrus detected |

| | |
|---|---|
| 202-216 | If all 30 gilts have not been recorded as in standing estrus during the |
| | activities described for d160-174 and d181-195, boar exposure will be |
| | repeated one final time following the procedure described above. |

TABLE 3
Serum progesterone concentrations by estrus
designation¹ in cycling and non-cycling gilts

| Estrus | Sample | Progesterone |
|---|---|---|
| designation | number | (ng/ml) |
| Silent | 2 | 14.9 ± 4.2 |
| Early | 3 | 18.6 ± 7.5 |
| Average | 3 | 23.3 ± 3.5 |
| Late | 2 | 21.2 ± 4.3 |
| Anestrus | 3 | Not detected |

¹Silent defined as detectable serum progesterone concentration without physical signs of standing estrus (eg. ears erect, swollen vulva, standing rigid to back pressure). Early, average, and late defined as identified physical signs of standing estrus at 160-181, 182-202, and 203-215 days of age, respectively. Anestrus defined as no physical signs of estrus or detectable serum progesterone by 215 days of age.

TABLE 4
Fold change in vaginal gene expression between gilts
expressing estrus early and late.

| | | | | Fold |
|---|---|---|---|---|
| | | Relative | | change: |
| | | expression | | early |
| Gene | Gene name/function | Early | Late | vs late |
| LOC110261636 | Unidentified | 36.52 | 0.054 | 612.35 |
| LOC100518075 | Unidentified | 1.35 | 0.003 | 370.59 |
| LOC100520618 | Unidentified | 0.393 | 0.307 | 31.14 |
| LIN28A | LIN28-homolog A; | 3.74 | 0.175 | 19.21 |
| | follicle growth and | | | |
| | maturation | | | |
| LOC100621931 | Unidentified | 0.158 | 0.014 | 15.05 |
| PRELID3B | PRELI domain containing | 2.92 | 0.109 | 24.46 |
| | 3B, phospholipid transfer | | | |
| | serine/threonine kinase | | | |
| STK32A | 32A; intracellular signal | 0.148 | 0.015 | 8.43 |
| | transduction | | | |
| ANO2 | Anoctamin 2; myometrial | 0.054 | 0.007 | 15.67 |
| | contractions | | | |
| LOXL2 | Lysyl oxidase-like 2; | 0.336 | 0.040 | 7.40 |
| | biogenesis of connective | | | |
| | tissue | | | |
| GYS2 | Glycogen synthase 2; | 0.013 | 0.059 | 0.200 |
| | Polycystic ovary | | | |
| | syndrome | | | |
| SPAI-2 | Sodium/potassium | 0.54 | 6.94 | 0.074 |
| | ATPase inhibitor SPAI-2 | | | |
| GTSF1 | Gametocyte specific | 0.049 | 0.548 | 0.074 |
| | factor 1; cell | | | |
| | differentiation and | | | |
| | spermatogenesis | | | |
| LOC100518848 | unidentified | 0.074 | 121.6 | 0.0005 |
| LBP | Lipopolysaccharide | 0.340 | 5.63 | 0.145 |
| | binding protein; immune | | | |
| | response | | | |
| | growth regulation by | 0.089 | 0.224 | 0.406 |
| | estrogen in breast cancer | | | |
| | 1; required for human | | | |
| | endometrial stromal | | | |
| GREB1 | decidualization | | | |
| IFN-ALPHA-16 | interferon alpha-16; | 0.277 | 1.742 | 0.139 |
| | infectious and | | | |
| | autoimmune disease | | | |
| | pathogenesis | | | |

TABLE 5
Biomarker genes for prepubertal selection of gilts
based on expression at d100 and 130. Based on fold
changes and statistical difference, the following are
preferred biomarkers for determining age at estrus:
GYS2, IFN-A-16, LIN28A, ANO2, LOXL2, GREB1.

| | | Relative | | |
|---|---|---|---|---|
| | | expression | | |
| | | (RPLPO) | | Fold |
| | | Early | Late | change¹ |
| | | estrus | estrus | Early |
| Gene | Metabolic pathway | females | females | vs late |
| IFN- | Immune response; | Down | Up | 0.139 |
| ALPHA-16 | infectious and autoimmune | | | |
| | disease pathogenesis | | | |
| GYS2 | Glycogen synthesis | Down | Up | 0.200 |
| LIN28A | Follicle growth; tissue | Up | Down | 19.21 |
| | repair | | | |
| ANO2 | myometrial contractions | Up | Down | 15.67 |
| LOXL2 | uterine hypertrophy | Up | Down | 7.40 |
| GREB1 | Endometrial stromal | Down | Up | 0.406 |
| | decidualization | | | |

¹Fold change based on expression across all timepoints.

### DISCUSSION

In FIG. 1, the LIN28A gene was 15 to 35-fold greater in early estrus gilts at all 3 time points and read counts were significantly greater in early estrus gilts at d100 of age. LIN28A knockout rate had reduced ovarian weight, with lower number of primordial ovarian follicles. These females had a reduced germ cell pool size, which resulted in lower number of mature oocytes and decreased fertility.

The genes ANO2 and LOXL2 are associated with uterine function. Both are expressed at least 10-fold greater in early estrus gilts at d130 (FIG. 1). ANO2 is expressed in uterine smooth muscle and plays a role in uterine contractions; LOXL2-deficient mice developed uterine hypertrophy and uterine carcinoma.

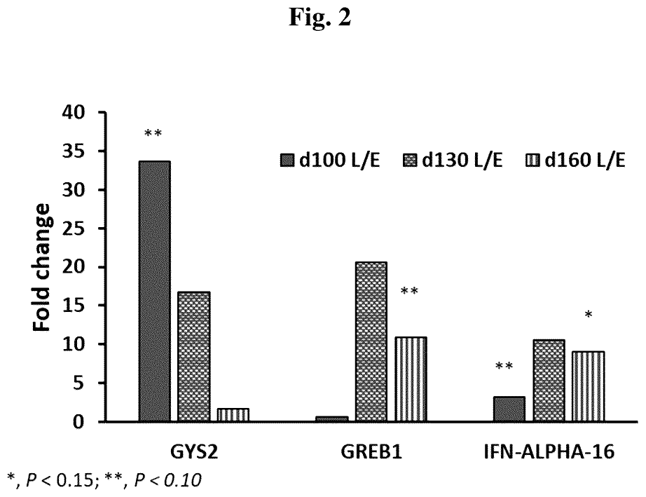

In FIG. 2, GYS2 (glycogen synthesis) gene was 35- and 15-fold greater in late estrus gilts at d100 and d130, respectively. Read count was significantly different at d100 and is associated with Polycystic ovary syndrome (PCOS) through its association with glycogen storage disease.

In FIG. 2, GREB-1 was expressed more than 10-fold greater in late estrus gilts. Read count was significantly greater at d160. In humans, GREB-1 is required for progesterone-driven human endometrial stromal cell decidualization. However, overexpression has been associated with estrogen-driven ovarian cancers.

In FIG. 2, IFN-alpha 16 was at least 5-fold greater in late estrus gilts at d130 and 160. Read count was significantly greater at d100 and tended to be greater at d160. IFN-alpha is a key cytokine in with anti-viral effects; however, overexpression or prolonged expression can modulate the development of autoimmunity or development of age-related diseases in HIV patients, respectively.

1. A method of determining whether estrus stage of a gilt at a certain age and acting accordingly, the method comprising:
a) obtaining vaginal epithelium cells from a gilt that is at least 80 days old,
b) identifying in the cells gene expression of at least one gene associated with growth, reproduction, or immune function in a gilt, and
c) determining estrus stage in the gilt, wherein a gilt which has undergone estrus is bred, and a gilt that has not yet undergone estrus is sold or culled.

2. The method of claim 1, wherein the gilt is at least approximately 130 days old.

3. The method of claim 2, wherein the gilt is under approximately 300 pounds.

4. The method of claim 3, wherein the gene expression is higher than a reference range of one or more genes from the group comprising: GYS2; IFN-alpha 16; LIN28A; ANO2; LOXL2; GREB1; LOC100518848; LOC110261636; LOC100518075; LOC100520618; PRELID3B; LOC100621931; GTSF1; STK32A; LPB; and SPAI2.

5. The method of claim 3, wherein gene expression is higher or lower than a reference range of one or more genes and reference range and one or more genes is selected from the group comprising:

| | Reference Range | | |
|---|---|---|---|
| Gene | (Ct) | Above | Below |
| LOC110261636 | 28-32 | Early | Late |
| LOC100518075 | 28-32 | Early | Late |
| LOC100520618 | 28-32 | Early | Late |
| LIN28A | 28-32 | Early | Late |
| LOC100621931 | 28-32 | Early | Late |
| PRELID3B | 28-32 | Early | Late |
| STK32A | 28-32 | Early | Late |
| ANO2 | 28-32 | Early | Late |
| LOXL2 | 28-32 | Early | Late |
| GYS2 | 28-32 | Late | early |
| SPAI-2 | 28-32 | Late | early |
| GTSF1 | 28-32 | Late | early |
| LOC100518848 | 28-32 | Late | early |
| LBP | 28-32 | Late | early |
| GREB1 | 28-32 | Late | early |
| IFN-ALPHA-16 | 28-32 | Late | early |

6. The method of claim 1, wherein the gilt is of an age selected from a group comprising: 80-160 days old; 90-140 days old; 100-130 days old; and 110-120 days old.

7. The method of claim 1, wherein gene expression is determined for 1 or more genes.

8. The method of claim 1, wherein gene expression is determined for at least 2 or more genes.

9. The method of claim 8, wherein gene expression is determined for at least 6 or more genes.

10. The method of claim 1, wherein early or late estrus is determined by comparing gene expression to a reference value for the gene at a relevant age range.

11. The method of claim 1, wherein early or late estrus is determined by comparing gene expression to an average range of values for the gene at a relevant age range.

12. The method of claim 1, which further comprises sorting the gilt into a group of gilts with the same early or late estrus determination.

13. The method of claim 12, which further comprises breeding a gilt determined to have early estrus.

14. The method of claim 12, which further comprises selling the gilt determined to have late estrus.

15. The method of claim 12, which further comprises selling a gilt determined to be anestrus.

16. The method of claim 15, wherein the gilt is sold before it reaches 300 pounds.

17. The method of claim 1, wherein gene expression levels identifies the gilt as having a relatively high reproductive potential.

18. The method of claim 1, wherein gene expression levels identifies the gilt as having a relatively low reproductive potential.

19. The method of claim 1, which further comprises identifying an additional marker of reproductive success selected from the group comprising: behavioral; hormonal; and physical.

20-40. (canceled)
